(19) United States
(12) Patent Application Publication
(10) Pub. No.: US 2024/0156580 A1
(43) Pub. Date: May 16, 2024

(54) Self-Supporting Delivery System for Extra-Oral Aerosol Reduction Device

(71) Applicant: Biothink Solutions LLC, Pepper Pike, OH (US)

(72) Inventors: Gary Golovan, Pepper Pike, OH (US); Bruce Golovan, Pepper Pike, OH (US)

(73) Assignee: Biothink Solutions LLC, Pepper Pike, OH (US)

(21) Appl. No.: 18/375,436

(22) Filed: Sep. 29, 2023

Related U.S. Application Data
(60) Provisional application No. 63/411,344, filed on Sep. 29, 2022.

(51) Int. Cl. A61C 17/06; A61G 15/10; A61M 1/00

(52) U.S. Cl. CPC A61C 17/096; A61G 15/10; A61M 1/84

(57) ABSTRACT

A self-supporting tube can support an associated medical or dental device, such as an extra-oral aerosol suction device. The self-supporting tube can be configured as a high-volume evacuator tube for a dental chair system. The self-supporting tube can include a first end that connects to an HVE port of a vacuum collection cannister directly or through a U-shaped tubular fitting and/or the first end of the self-supporting tube can connect to an inlet of a high-volume evacuator valve, and the self-supporting tube can include an opposite second end that connects to the associated aerosol suction device. The self-supporting tube can include at least one lumen that fluidically connects the first and second ends. The self-supporting tube can further include a manually operated inline valve. The self-supporting tube can be disposable and can be formed into a desired shape. When connected to an HVE valve, the self-supporting tube can include an anti-rotational device. The self-supporting tube can be provided as part of a modular system including at least one flexible tube segment and the self-supporting tube connected together. The self-supporting tube can be connected to an associated support structure of a dental delivery system unit such as an assistant's chair by a sleeve. The self-supporting tube can be connected to a support arm extending from a stopper connected to an HVE valve cradle. The self-supporting tube can include an aerosol suction funnel formed as a one-piece structure with the remainder of the self-supporting tube.

### CROSS-REFERENCE TO RELATED APPLICATION

This application claims priority from and benefit of the filing date of U.S. provisional application Ser. No. 63/411,344 filed Sep. 29, 2022, and the entire disclosure of said provisional application is hereby expressly incorporated by reference into the present specification.

### BACKGROUND

It has become increasingly important to remove potentially harmful extra-oral bio-aerosols from dental procedure environments for the safety of the dentist, staff, and patients. FIG. 1 shows a known system for supporting an extra-oral suction device 101 in the vicinity of the mouth located in a dental chair 102. An inner end of an adjustable support arm 103 is connected to the chair 102, typically adjacent the headrest 104 and an opposite, outer end of the arm 103 includes a clip, clamp, or other retention device 105 that receives and retains a dental or other medical device such as the illustrated suction device 101. The support arm 103 is self-supporting and holds the position of device 105 on the outer end thereof (and the suction device 101 or other medical device held thereby) can be selectively and manually positioned in a desired location relative to the dental chair 102 and patient located in the chair. The suction device 101 is also operably connected to a suction conduit such as a High-Volume Evacuation Tube (HVET) 106 that is connected to a suction source typically through a Vacuum Collection Canister (VCC) associated with the chair 102.

The system in FIG. 1 and similar arrangements are suboptimal for many reasons. The support arm 103 is a separate device that must be purchased, stored, cleaned, and otherwise accounted for. The presence of the arm 103, in addition to the HVET 106 can interfere with the dental procedure being performed in the somewhat confined space available, and adjustment of the position of the arm 103 (and the suction device 101/HVET supported thereby) can be cumbersome and time-consuming due to the fact that the dental practitioner must consider the position of both the HVET 106 and the arm 103, along with the position of the suction device 101. The current setups for a mounted arm 103 to hold a suction device 101 for extra oral aerosols are cumbersome, get in the way of the dentist, dental assistant, and the patient. They are time consuming to set up and tear down, clean, maintain, not easily moved from room to room, and are expensive, etc. As such, a need has been identified for a new and improved system for overcoming these deficiencies while providing superior results.

### SUMMARY OF THE INVENTION

A self-supporting tube can support or hold an associated medical or dental device, such as an extra-oral aerosol suction device.

In one variation, the self-supporting tube can be configured as a high-volume evacuator tube for a dental chair system.

In another variation, the aerosol suction device can include a particle capturing media.

In a further variation, the self-supporting tube can include a first end that connects physically and fluidically to an HVE port of a vacuum collection cannister and/or to an inlet of a high-volume evacuator valve, and the self-supporting tube can include an opposite second end that connects physically and fluidically to the associated device such as the aerosol suction/filter device, and the self-supporting tube can include at least one lumen that fluidically connects the first and second ends.

In another variation, the self-supporting tube can further include a manually operated inline valve.

In a further variation, when connected to a high-volume evacuator valve, the self-supporting tube can include an anti-rotational device that prevents rotation of the self-supporting tube relative to an associated cradle in which the high-volume evacuator valve is situated.

Another variation of the self-supporting tube can be provided as part of a modular system including at least one flexible tube segment and the self-supporting tube connected together, and at least one of the flexible tube segment and the self-supporting tube can be connected to an associated support structure of a dental delivery system unit by a flexible sleeve through which said self-supporting tube passes, wherein the support structure may be any one or more of: (i) a dental patient chair; (ii) a dental assistant's chair; (iii) a dentist chair; (iv) a lighting unit; (v) a cart or table; (vi) an imaging unit; or another structure.

In another possible variation, the self-supporting tube can be connected to an associated support structure of a dental system delivery unit by a flexible sleeve through which said self-supporting tube passes.

In another variation, the support structure can comprise at least one of: (i) a dental patient chair; (ii) a dental assistant's chair; (iii) a dentist chair; (iv) a lighting unit; (v) a cart or table; (vi) an imaging unit; or another structure.

In another variation, the self-supporting tube can be made entirely from a disposable material.

In another variation, the self-supporting tube can include: (i) a first end adapted to be connected to a suction source such as a vacuum collection cannister or a high-volume evacuator valve; and (ii) a second end that comprises the associated extra-oral aerosol suction device, wherein the extra-aerosol suction device is defined from paper or cardboard or another material.

In another variation, the extra-aerosol suction device can be constructed as a one-piece structure with the self-supporting tube.

In a further variation, the self-supporting tube can include an adhesive or other fastening element on an exterior surface for adhering said tube to at least one of: (i) a dental patient chair; (ii) a dental assistant's chair; (iii) a dentist chair; (iv) a lighting unit; (v) a cart or table; (vi) an imaging unit; or another structure.

In an additional possible variation, the self-supporting tube can include a flat exterior surface on which an adhesive or other fastening element is located.

In a further possible variation, the self-supporting tube can comprise a non-circular cross-section with at least one flat exterior surface on which an adhesive or other fastening element is located for selectively connecting the tube to at least one of: (i) a dental patient chair; (ii) a dental assistant's chair; (iii) a dentist chair; (iv) a lighting unit; (v) a cart or table; (vi) an imaging unit; or another structure.

In an additional possible variation, a first end of the self-supporting tube may be connected to a vacuum collection cannister through a U-shaped connector tube fitting comprising a first end connected to a port of the vacuum collection cannister and a second end connected to the first end of the self-supporting tube, wherein an opposite second end of said self-supporting tube includes an extra-oral aerosol suction device.

In a further possible variation, a first end of the self-supporting tube can be connected to an inlet of a high-volume evacuator valve that is supported in a cradle and can include an extra-oral aerosol suction device located at a second end of said self-supporting tube.

In another possible variation, an anti-rotation device may be operably engaged between the self-supporting tube and the cradle to prevent undesired rotation or movement of the self-supporting tube.

In a further possible variation, the self-supporting tube is connected to a support arm that projects outwardly from a stopper that is non-rotatably seated in an HVE valve cradle.

In another variation, the self-supporting tube can be provided as part of a modular system including a tube segment supported on a dental patient's chair or a dental assistant's chair, and the tube segment may include first and second fittings on opposite first and second ends thereof, and first and second end caps may be optionally releasably engaged with the first and second fittings of the tube segment.

### BRIEF DESCRIPTION OF THE DRAWINGS

FIG. 1 (Prior Art) shows a known system for supporting an extra-oral suction device.

FIG. 2 shows a dental delivery system unit including a self-supporting delivery system for an extra-oral aerosol reduction device according to a variation of the present development.

FIG. 3 illustrates a vacuum collection cannister connected to a self-supporting delivery system for an extra-oral aerosol reduction device according to one variation of the present development.

FIG. 4 (Prior Art) shows a bottom view of the cannister portion of FIG. 3.

FIG. 5 shows a self-supporting delivery system for an extra-oral aerosol reduction device according to another variation of the present development.

FIG. 6 shows a self-supporting delivery system for an extra-oral aerosol reduction device according to another possible variation of the present development.

FIG. 7 shows a self-supporting delivery system for an extra-oral aerosol reduction device according to another possible variation of the present development including an anti-rotation device.

FIG. 8 shows a self-supporting delivery system for an extra-oral aerosol reduction device according to another possible variation of the present development.

FIG. 8A shows aspects of a self-supporting delivery system for an extra-oral aerosol reduction device according to further possible variations of the present development.

FIG. 8B shows a support sleeve for further a possible variation of the present development.

FIG. 9 shows a kit for a modular self-supporting delivery system for an extra-oral aerosol reduction device according to a further possible variation of the present development.

FIG. 10 shows a dental assistant's chair including a self-supporting delivery system for an extra-oral aerosol reduction device according to a further possible variation of the present development.

FIG. 11 shows a self-supporting delivery system for an extra-oral aerosol reduction device according to another possible variation of the present development.

FIG. 12A shows a self-supporting delivery system for an extra-oral aerosol reduction device according to another possible variation of the present development.

FIG. 12B shows a self-supporting delivery system for an extra-oral aerosol reduction device according to another possible variation of the present development.

### DETAILED DESCRIPTION OF THE INVENTION

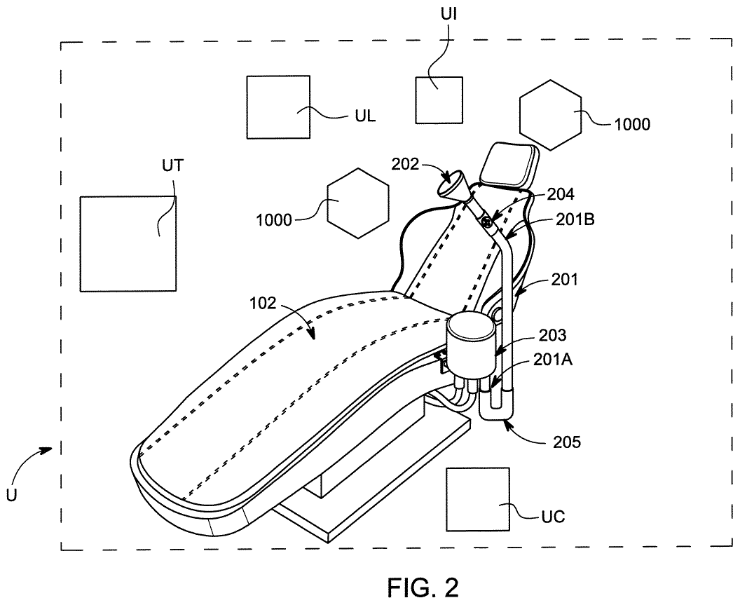

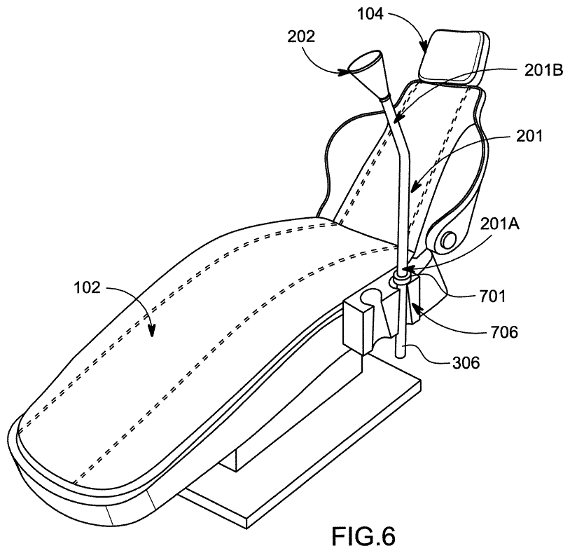

According to an embodiment of the present development, with reference to FIG. 2, a self-supporting tube 201 that is supporting a suction a device 202, such as an extra-oral Aerosol Suction Device (ASD) or other device, is provided and configured for supporting the suction device 202 in a desired position relative to a dental chair 102 and a patient located therein. The dental chair 102 can be part of a dental delivery system unit U that can comprise the patient chair 102, auxiliary chairs 1000 for the dentist and dental assistant, a lighting unit UL, an imaging unit UI, one or more tables UT, one or more fixed or mobile carts UC, and various other components used by the dentist and the dental assistant for delivering dental procedures. The self-supporting tube 201 includes a first end 201A adapted to be physically and fluidically connected to a suction source or vacuum source and includes a second end 201B that is spaced from the first end 201A and adapted to be connected to a suction device 202 to be operatively supported or held in the vicinity of the dental chair 102. As described in more detail below, the self-supporting tube 201 includes at least one lumen 201L (FIG. 5) or fluid (vacuum) passage that opens through the opposite first and second ends 201A, 201B and that extends between and fluidically connects the first and second ends 201A, 201B. The device 202 such as the illustrated extra-oral ASD can optionally comprise a particle capturing media located therein or thereon that removes pathogens and/or other particulate contaminants from the air that enters the ASD device 202 before the air enters self-supporting tube 201.

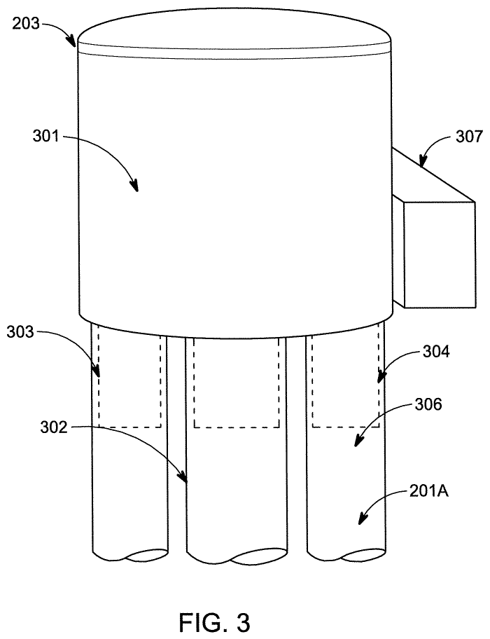

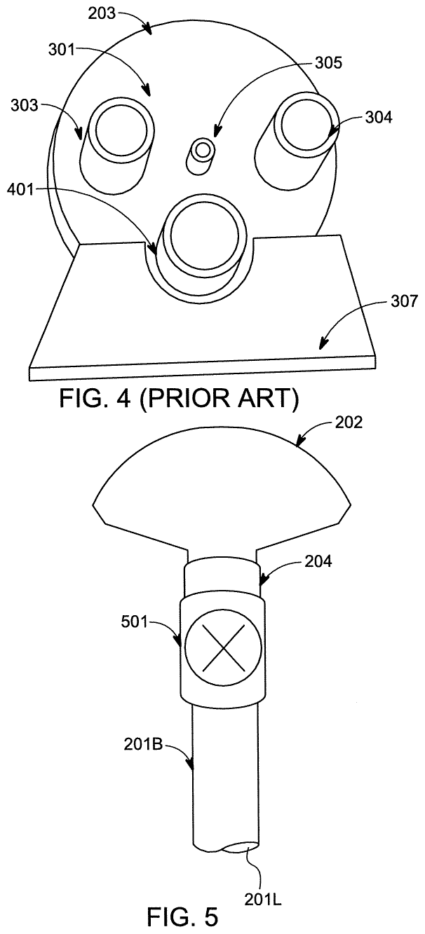

In the embodiment of FIG. 2, the first end 201A of the self-supporting tube 201 is operatively connected to a high-volume evacuator port of a Vacuum Collection Canister (VCC) 203, associated with the chair 102. As shown in the example of FIG. 2, the first end 201A of the self-supporting tube 201 includes an optional U-shaped tube fitting 205 that connects at a first end to the VCC 203 and that connects at a second, opposite end to the self-supporting tube 201. The U-shaped fitting 205 can non-rotatably connect to the VCC 203 to prevent undesired movement of the self-supporting tube 201 and the ASD device 202. One non-limiting example of a VCC 203 is shown in FIG. 3 (a side view) and FIG. 4 (a bottom view). As shown in FIG. 3 and FIG. 4, the VCC includes a body 301 with an internal hollow space. The body 301 includes a main outlet port 401 adapted to be fluidically connected to a vacuum source (such as a remotely located dental suction motor) through a main suction tube or main suction conduit 302. As best seen in the bottom view of FIG. 4, the VCC 203 includes at least two inlet ports such as a first High-Volume Evacuator (HVE) port 303, a second HVE port 304, and a Saliva Ejector (SE) port 305 that are each in fluid communication with the main outlet port 401 through the internal space of the VCC 203 (and thus in fluid communication with the vacuum motor or other vacuum source connected to the main outlet port 401). In one arrangement, the SE port 305 is fluidically connected to an intra-oral saliva ejector (not shown) through flexible tube, and the first high volume evacuator port 303 is fluidically connected to an intra-oral suction device (not shown) through a flexible tube such as a first HVE tube.

According to an embodiment of the present disclosure, the first end 201A of the self-supporting tube 201 is operatively physically and fluidically connected to the second high-volume evacuator port 304 such as by a first fitting 306 connected to the first end 201A of the self-supporting tube 201 and that that frictionally engages or otherwise mates with the second HVE port 304 in a fluid-tight manner. Alternatively, the first end of 201A of the self-supporting tube 201 can be conformed and dimensioned to frictionally or otherwise mate directly with the second HVE port 304 in a fluid-tight manner. The opposite, second end 201B of the self-supporting tube 201 can include a second fitting 204 that mates with the extra-oral suction device 202 in a fluid-tight manner with a friction fit or otherwise. In another example, the U-shaped tubular fitting 205 is non-rotatably connected at a first end to the high-volume evacuator port 304 of the VCC 203 and the U-shaped fitting is rotatably or non-rotatably connected at its opposite second end to the first end 201A of the self-supporting tube 201, in which case the U-shaped fitting provides a robust connection between the first end 201A of the self-supporting tube 201 and the second HVE port 304 and also limits stress exerted on the VCC 203 in the region of the HVE port 304.

As shown in FIG. 5, the second fitting 204 located at the outer end of the self-supporting tube 201 can optionally include a manually actuated inline valve 501 placed at any point between the extra-oral suction device 202 and the tube second end 201B that can be selectively manually operated to be configured in a first or opened position for fluid communication of the suction stream between the device 202 and the internal passage(s) of self-supporting tube 201 and a second or closed position in which the inline valve 501 blocks fluid communication of the suction stream between the device 202 and the internal passage(s) of the self-supporting tube 201 when the suction is not desired, such as to reduce noise. The dentist and/or assistant can selectively open and close the valve 501 as required.

Referring again to FIG. 3 and FIG. 4, the VCC 203 can be connected to the dental chair 102 by a bracket 307, but it can be separated from the chair 102 and connected to another support structure in the vicinity of the chair 102 and/or associated with the chair 102 and/or an associated support structure such as the workstation of the dentist or an assistant. Of course, the self-supporting tube 201, itself, can have any desired or required length or diameter as required for a particular application and as needed to mate with the HVE port 304 of VCC 203 or other suction source at the first end 201A and with the device 202 at the opposite end 201B.

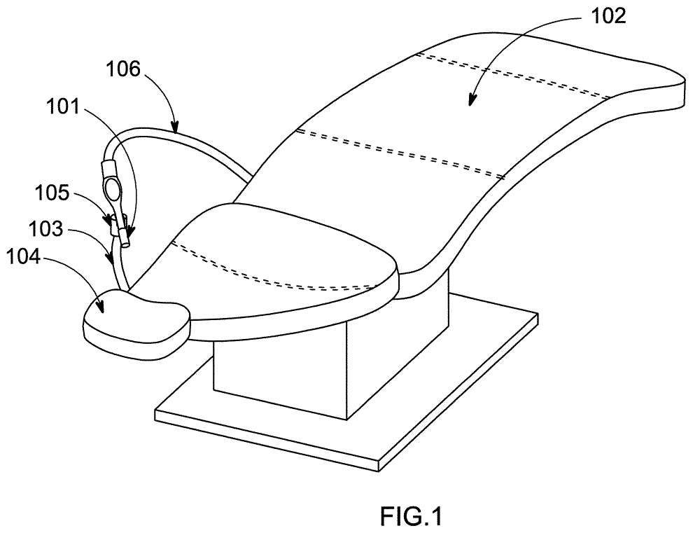

As noted, the second end 201B of the self-supporting tube 201 is operatively connected to the extra-oral suction device 202 (such as the aerosol suction device ASD) such that the device 202 is physically and fluidically connected to the self-supporting tube 201. The self-supporting tube 201 is sufficiently rigid to immovably support the weight of the device 202 without use of any separate support arm (such as the support arm 103 of FIG. 1) or any other support aid. Thus, a dental practitioner can selectively, move and/or form the self-supporting tube 201 to be located in any one or more shapes and/or locations between the opposite first and second tube ends 201A, 201B to alter the operative position of the device 202 as necessary before, during, and after a dental procedure. It should be noted that the operative position of the self-supporting tube 201 can be easily adjusted using minimal effort by the dental practitioner which is convenient and safe for the medical practitioner and patient. This configuration, without the use of a separate support arm 103 as shown in FIG. 1, provides an advantage of freeing up space within the operative field for the dentist and dental assistant to more optimally perform dental procedures.

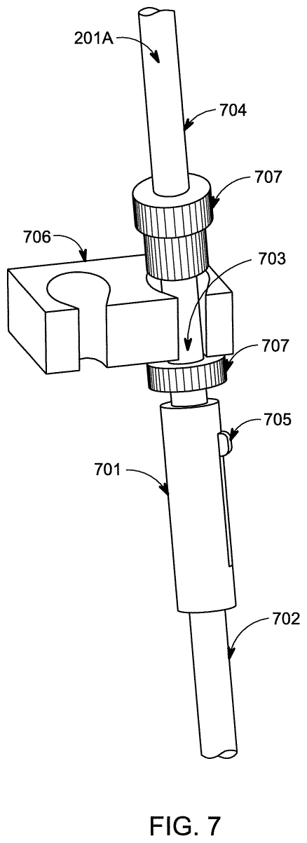

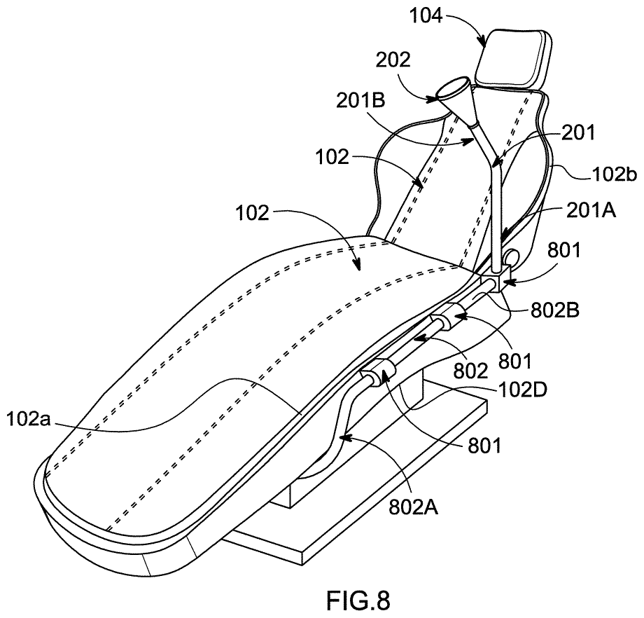

FIG. 6 and FIG. 7 illustrate another embodiment of a self-supporting tube 201 for supporting an extra-oral aerosol suction device 202. Except as otherwise shown and or described herein, the tube 201 of FIG. 6 and FIG. 7 is of an identical design, but in a different configuration to the tube 201 of FIG. 2, FIG. 3 and FIG. 4. In particular, the self-supporting tube 201 of FIG. 6 and FIG. 7 includes a first end 201A that is adapted to mate with a conventional hand-held high-volume evacuator (HVE) valve 701 that is operatively connected to a suction source such as the second HVE port 304 of the VCC by a conventional flexible HVE suction tube/suction conduit 702. The HVE valve 701 has an open circular inlet 703 (FIG. 7) that typically has an 11 millimeter (mm) or a 15 millimeter (mm) inside diameter and the first end 201A of the self-supporting tube 201 includes a first fitting 704 that is adapted to be received within and frictionally mate with the open inlet 703 of the HVE valve 701 such that the internal passage of the self-supporting tube 201 is in fluid communication with the HVE valve 701.

The HVE valve 701 also includes a hand-operated on/off actuator 705 that can be selectively configured to be in either a first position to open the HVE valve 701 for fluid communication of the valve inlet 703 with the suction motor or other vacuum source by way of the flexible suction tube/conduit 702 or a second or closed position to close the HVE valve 701 to block fluid communication between the valve inlet 703 and the suction source by way of the flexible suction tube/conduit 702 as needed, e.g., to reduce noise.

The length of the self-supporting tube 201 will depend on installation and relative position of the vacuum source and where the dental professional wants to position the extra-oral suction device 202. The position of the VCC 203 and/or the HVE valve 701 will influence the path of the self-supporting tube 201.

As also shown in FIG. 6 and FIG. 7 the HVE valve 702 is selectively releasably received and retained by a cradle 706 that can be connected to or otherwise located in the vicinity of the chair 102 or that can be connected to another support structure such as a workstation for the dentist or the assistant. To prevent undesired relative rotation of the HVE valve 701 relative to the cradle 706 when the self-supporting tube 201 is connected thereto, the HVE valve 701 can include a knurled or textured body 703 where it abuts the cradle 706. Alternatively or additionally, the first fitting 704, the HVE valve 701, and/or other part of the self-supporting tube 201 can include an anti-rotation device 707 that is operably engaged between the self-supporting tube 201 and the cradle 706 or other structure to prevent undesired rotation or movement of the self-supporting tube 201. The anti-rotation device 707 can be non-rotatably connected to the first fitting 704, the HVE valve 701, and/or other part of self-supporting tube 201 and that projects outwardly and abuts or otherwise engages the cradle 706 (as shown in FIG. 7) and or that abuts another fixed location on the chair 102 or other structure to prevent rotation of the HVE valve body 703 about its longitudinal axis relative to the cradle 707.

It is reiterated here that a self-supporting tube 201 provided in accordance with the present development can also be used to support other devices 101 at the second end 201B thereof, instead of and/or in addition to the aerosol suction device ASD, and such alternative devices 202 can include but are not limited to a mirror, a camera, a microphone, a video display, a mobile electronic device, a light source, a tray or container, and/or any other device required or desired by a dental practitioner or other medical practitioner or by the patient being treated.

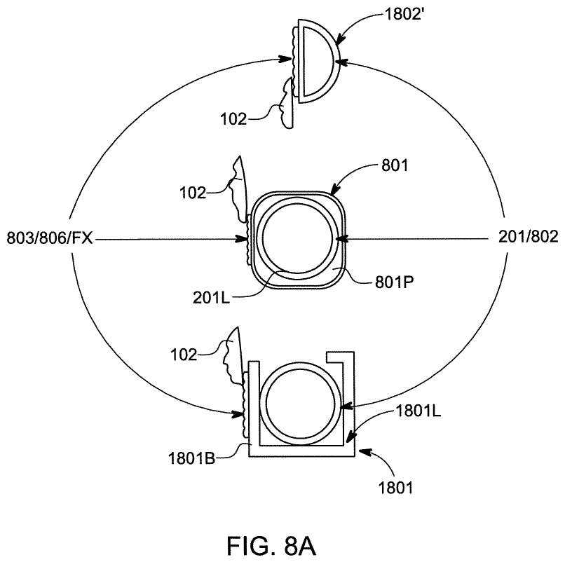

As shown in FIG. 8, the dental chair 102 can include one or more tube retainers such as one or more tube support sleeves 801 that are connected to the chair 102 and that selectively receive and retain a segment of the self-supporting tube 201 or that receive and retain a segment of conventional flexible tubing 802 as shown. A first end 802A of the conventional tubing 802 is connected in fluid communication with the VCC 203 or other vacuum source and an opposite second end 802B of the conventional tubing 802 is connected in fluid communication with the end 201A of the self-supporting tube 201. The one or more sleeves 801 can be connected to the seat 102a and/or the back 102b and/or the headrest 104 of the chair 102 or in a region under the underside 102d of the chair 102 and or behind the back 102b of the chair 102.

FIGS. 8A and 8B partially illustrates a dental chair 102 comprising at least one tube support sleeve 801 connected thereto by a fastening element such as an adhesive, a hook-and-loop type (e.g., VELCRO® brand) fastening element, or other fastening element 803. Alternatively, the tube support sleeve 801 can be sewn or otherwise manufactured as part of the chair 102. The tube support sleeve 801 defines a channel or passage 801P through which the self-supporting tube 201 (or alternatively a conventional tube 802) extends. The tube support sleeve 801, itself, can optionally include first and second portions 801A,801B (FIG. 8B) that can be selectively connected to each other and disconnected from each other, such as by using a hook-and-loop type (e.g., VELCRO® brand) fastening element 801C, in order to be separated and then reconnected to receive and retain the self-supporting tube 201 or a conventional tube 802. Alternatively, the tube 201 or 802 can be axially fed through the passage 801P. The sleeve 801 can be defined from a natural or synthetic fabric or other flexible material such as vinyl, leather, cloth, and/or a polymeric sheet or laminate of one or more of the foregoing materials and/or others. One or more of the tube support sleeves 801 can be connected under the underside 102d of the chair 102 and or behind the back 102b of the chair 102 to be further out of the way and out of sight. The tubing support sleeve(s) support the tubing 201,802 and thus reduce stress on the connected components such as the port 304 of the vacuum collection canister 203.

FIG. 8B also shows that the tube support sleeve 801 can be used to support a tube segment 802′ that can be a segment of conventional flexible tube 802 or a segment of self-supporting tube 201. The tube segment 802′ can comprise first and second tube fittings 802F1,802F2 on its opposite first and second ends. In one example, a conventional tube 802a that is in fluid communication with the HVE port 304 of the VCC 203 is frictionally or otherwise releasably connected to the first tube fitting 802F1 and the first end 201A of the self-supporting tube 201 (optionally including the ASD 202 on its opposite end 201B) is connected to the second tube fitting 802F2. In this manner, the tube segment 802′ can remain connected to the dental chair 102 and the tubes 802a,201 selectively connected to and disconnected from the tube segment 802′ as desired for a particular patient or procedure. Optionally, a plug or end cap EC can be connected to the first and/or second fittings 802F1,802F2 of the tube segment 802′ when the tube segment is not in use to seal tubing segment 802′.

FIG. 8A also illustrates an example of a tube support sleeve 801 comprising an adhesive 806 such as a releasable and reusable “on/off” adhesive or other fastening device or element that allows the sleeve 801 to be adhered to any desired structure or location within the dental delivery system unit U such as the dental patient's chair 102, an auxiliary chair 1000 such as the dental assistant's chair or the dentist's chair, a lighting unit UL, imaging unit UI, a mobile cart UC, a table UT, and/or any other structure as desired to provide a stable path for the tubes 201,802,802′. The adhesive 806 can comprise a strong yet releasable and reusable “on/off” adhesive that allows the sleeve 801 to be removed and repositioned on a chair or other support structure multiple times before the adhesive must be replaced. The adhesive can be replaced by another fastening element such as a hook and loop type fastening element or the like.

FIG. 8A also shows an optional tubing segment 1802′ that is similar to the tubing segment 802′ shown in FIG. 8B, except that it comprises at least one external flat side F (such as the illustrated D-cross-section) that includes a fastening element FX thereon such as an adhesive, snaps or other fasteners, or the hook or loop component of a hook-and-loop fastening element such that the tubing segment 1802′ can be selectively fastened or adhered or otherwise selectively connected to any desired support structure in the dental delivery system unit U such as the patient's chair 102, the auxiliary chair 1000 for the dentist or the assistant, a workstation, a cart, and/or any other desired location. In the embodiment where the fastening element comprises an adhesive, a strong yet releasable and reusable “on/off” adhesive can be used so that the tubing segment 1802′ can be removed and repositioned multiple times before the adhesive must be replaced. As with the tube segment 802′ of FIG. 8B, the tube segment 1802′ can also include first and second connectors 802F1,802F2 (see FIG. 8B) on its opposite ends for mating with other tubes such as the flexible tube 802 and a self-supporting tube 201. Because the tubing segment 1802′ includes an adhesive or other fastening element FX for being directly connected to the patient's chair 102, the auxiliary chair 1000 for the dentist or the assistant, a workstation, a cart, and/or any other desired location in the dental delivery system unit U, it does not require any additional sleeve 801 to be fixed in a desired location. In addition, the presence of the external flat side F maximizes the surface area for connection to the chair 102 or assistant's chair 1000 or other support location and improves cleanliness by preventing aerosols and other contaminants from entering the region between the tube 1802′ and the chair 102,1000.

As also shown in FIG. 8A, one or more of the tube support sleeves 801 can be provided as a clip 1801 of any desired axial length and defined from a polymeric and/or metal material and comprising at least one resilient leg 1801L that captures the tubing 201,802,802′ adjacent a base 1801B. The base 1801B can comprise a fastening element 803,806 on an exterior or rear surface thereof, such as an adhesive, snaps or other fasteners, or the hook or loop component of a hook-and-loop fastening element, such that the clip 1801 can be connected to any desired support location in the dental delivery system unit U such as the patient's chair 102, auxiliary chair 1000, or any other desired location such as a mobile cart UC, table UT, or a wall structure.

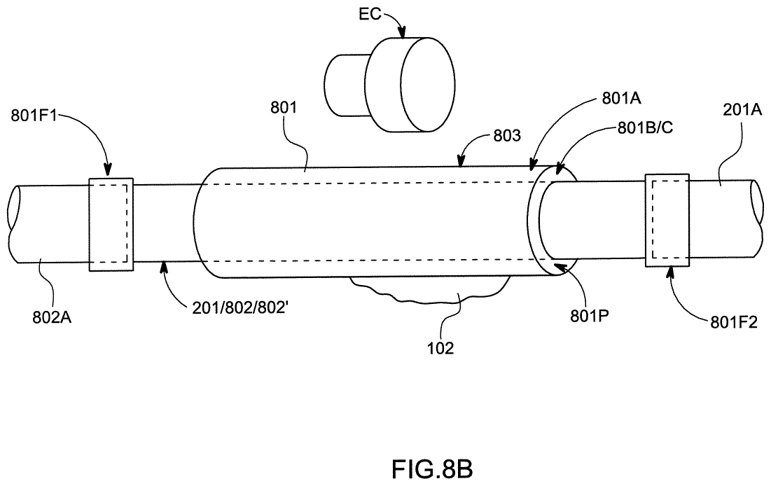

As shown in FIG. 9, the present system can comprise a group or kit of modular components that can be assembled as described above and otherwise to create a self-supporting delivery system for an extra-oral aerosol reduction device. The system can comprise an extra-oral aerosol suction device 202, an optional valve 501, at least one segment of formable self-supporting tubing 201, optionally at least one conventional flexible tube segment 802, optionally a U-shaped tubular fitting 205, and optionally at least one flexible tube support sleeve 801 or other tube retainer.

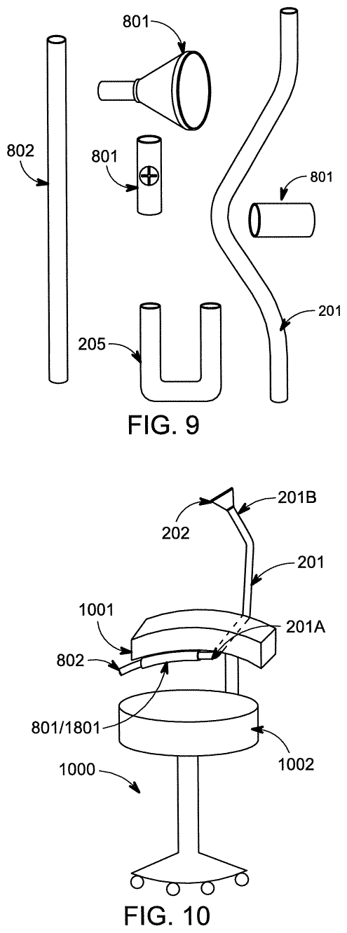

FIG. 10 shows an auxiliary chair 1000 that can be a dental assistant's chair or a dentist chair including an optional armrest 1001 and a seat 1002. The chair 1000 is typically provided nearby the patient's chair 102 and is used by the dentist or by the dentist's assistant. In accordance with one alternative embodiment of the present development, the armrest 1001 (as shown) or seat 1002 or any other part of the chair 1000 includes a tube support sleeve 801 or 1801 as described above in relation to FIGS. 8A and 8B for selectively retaining and supporting the self-supporting tube 201, the flexible tube 802 and/or a tube segment 802′. The chair 1000 provides a good location for routing the tubes 201,802, 802′ and, importantly, allows the tubes 201,802,802′ supported by the sleeve 801 to move together with the chair 1000 so that the chair 1000 and/or the person occupying the chair 1000 do no become entangled in the tubes 201,802,802′ when the chair 1000 is moved before, during, and/or after a procedure. As noted, the auxiliary chair 1000 can be dental assistant's chair or the chair used by the dentist. FIG. 10 also illustrates a variation of the present development in which a segment of the self-supporting tubing 201 is fixedly secured to the armrest 1001 and/or the seat 1002 using one or more of the sleeves 801 or clips 1801 and the self-supporting tube 201 projects outwardly away from the chair 1000 to its second end 201B that includes the extra-oral aerosol reduction device 202 connected thereto or defined as a part thereof. The sleeve(s) 801 and/or clip(s) 1801 can be connected to the armrest 1001 and/or other part of the chair 1000 using any suitable fastening device such as adhesive, a hook and loop type fastening element, sewn stitching, snaps, and/or other fasteners. In one example, the sleeve(s) 801 is sewn into and provided as part of the fabric or other upholstery covering of the chair seat 1002 or armrest 1001. As described above, the tube 201 is self-supporting and can be shape formable such that the extra-oral aerosol reduction device 202 can be positioned at any desired orientation or location by manually bending the tubing 201. The first end 201A of the tubing 201 can be in fluid communication with the VCC 203 directly or through one or more intermediate tube segments of self-supporting tubing 201 and/or conventional flexible tubing 802 and/or the U-shaped tube fitting 205. The valve 501 can optionally be included at any location along the self-supporting tubing 201, tubing 802, or as part of the U-shaped tubular fitting 205.

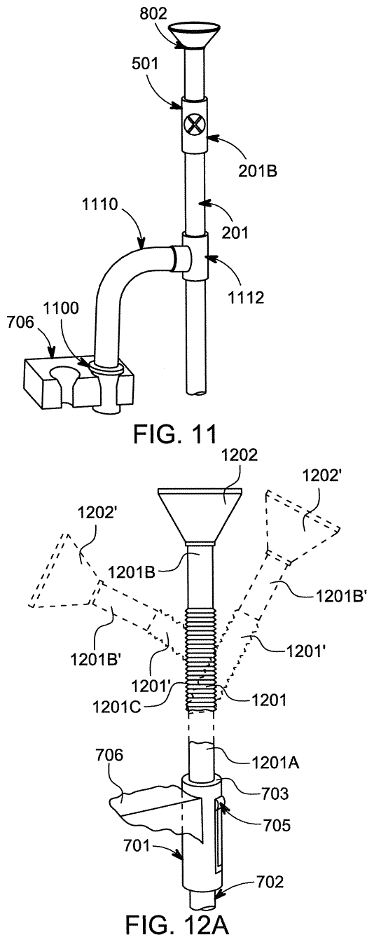

FIG. 11 shows another alternative design wherein a rubber or similar resilient stopper 1100 is engaged with the HVE valve cradle 706 with a pressure or friction fit such that the stopper 1100 does not rotate relative to the cradle 706. The stopper 1100 comprises a support arm 1110 that projects outwardly therefrom and that includes a selectively engageable and releasable clamp 1112 on its outer end. The clamp 1112 can be secured to the self-supporting tubing 201 that includes extra-oral aerosol reduction device 202 connected thereto (and optionally also the valve 501) at its outer end 201B.

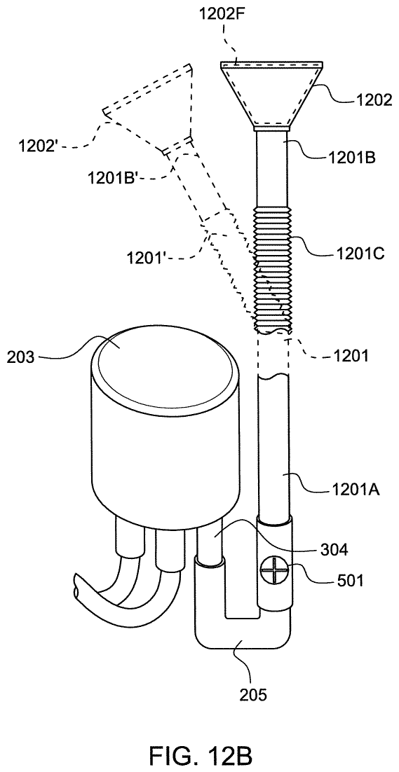

In an alternative variation, all of the described version of the self-supporting tube 201 can be manufactured from a paper, cardboard, or other cellulosic pulp-based material (all generally referred to herein as “paper” which is intended to encompass paper, cardboard, and other cellulosic pulp-based materials) or a polymeric material or other suitable material that is disposable. Such a disposable tube 201 can be any desired length and be shape-formable or can be manufactured with a select shape that can be straight between its opposite ends 201A,201B or that can include one or more linear and/or curved portions that to define a non-linear path between the opposite ends 201A,201B. FIG. 12A shows one such variation where the self-supporting tube 1201 that is similar to the tube 201 described above except that it is defined as one-piece from a disposable paper or a disposable polymeric material. A first end 1201A of the tube 1201 is connected to the inlet 703 of the hand-held high-volume evacuator (HVE) valve 701 that is supported in the valve cradle 706, and an opposite outer or second end 1201B can be plainly cylindrical or, in the illustrated variation, can include an aerosol intake funnel 1202 defined at the second end 1201B that is part of the one-piece tube structure 1201 or attached thereto and that functions as an extra-oral aerosol suction device (ASD) 1202 similar to the separate ASD 202. The self-supporting tube 1201 is disposable. As noted, the extra-oral aerosol suction device 1201 can be omitted in one variation. The tube 1201 can include pleats or corrugations 1201C that allow the tube 1201 to be manually shape-formed to locate the tube 1201, its second end 1201B, and aerosol suction device 1202 at any desired location as shown in broken lines respectively at 1201′, 1201B′ at 1202′.

FIG. 12B is similar to FIG. 12A but shows that the first end 1201A of the self-supporting disposable tube 1201 can be connected to the suction port 304 of the vacuum collection cannister 203 through the U-shaped tubular fitting 205, with a first end of the tubular fitting 205 connected to the suction port 304 of the vacuum collection cannister 203 and the opposite second end of the U-shaped fitting 205 connected to the first end 1201A of the self-supporting tube 1201. Optionally, the on/off valve 501 described above can be included in the U-shaped tubular fitting 205 or connected in-line between the U-shaped tubular fitting 205 and the first end 1201A of the tube 1201. Alternatively, the first end 1201A of the self-supporting disposable tube 1201 can be connected directly to the suction port 304 of the vacuum collection cannister 203. The tube 1201 can include pleats or corrugations 1201C that allow the tube to be manually shape-formed to locate the tube 1201, its second end 1201B, and aerosol suction device 1202 at any desired location as shown in broken lines respectively at 1201′, 1201B′ at 1202′.

A disposable tube 1201 can be disposable for each patient and is lightweight and may not need any anti-rotation device when used with the HVE valve 701 in the cradle 706. The disposable tube 1201 is fabricated so it is self-supporting and can be formable by including corrugations or a metal wire embedded or connected along all or part of its length that can be bent and hold its shape or another material that holds its shape. The disposable tube 1201 is inexpensive to make and buy. An optional filter 1202F can be included on or in the have a filter in the intake funnel 1202 such as a cone filter that fits within and conforms to the shape of the funnel or that elastically or otherwise stretches over the outer rim of the funnel 1202.

The self-supporting tube 201 can be constructed from any suitable polymeric and/or metallic materials or, as noted above, can be constructed from paper or cardboard or other cellulosic pulp based disposable material. In one variation, the self-supporting tube can be formable and re-formable into a desired shape and will retain such shape, while in another variation the tube 201 can be manufactured with a select linear or non-linear shape. In one example, the tube 201 is constructed from one or more pieces of polymeric or “plastic” tubing that comprises one or more sections that are corrugated or pleated (see 1201C in FIG. 12B) to provide for shape-holding formability, and such corrugated or pleated section(s) can extend for part of or along the entire axial length of the self-supporting tube 201,1201. In another example, a polymeric film can be formed into the desired self-supporting tube 201,1201. The tube 201,1201 can be disposable and in certain variations can be biodegradable.

Use of the sleeve(s) 801 and/or clip(s) 1801 and/or other tube retainers as described herein allows the self-supporting tubing 201 and/or other tubing 802 to be used more effectively, conveniently, and safely with any style of dental delivery system such as front, side, or rear delivery, because the tubes 201,802 are routed to areas where the patient, dentist and/or assistant will not become entangled or encumbered by the tubing 201,802. Furthermore, the presence of the sleeve(s) 801 and/or clips 1801 ensures that the tubing 201,802 held thereby will move together with the chair 102 when the position thereof is adjusted before, during, and/or after a procedure, or when the assistant's or other auxiliary chair 210 is moved. Incorporating a Flexible Sleeve 801 mounted on the underside of the dental chair 102 (or any part of the dental chair) removes part of the setup to an out of the way location. This frees up part of the area above the chair 102 providing for a less obtrusive working environment for the dental team and the patient. The semi-rigid arm (tubing) 201 and flexible sleeve 801 can be divided into sections that can be different lengths to accommodate different dental configurations of the dental delivery systems (front, side, rear and mobile carts, etc.). Mounting the flexible sleeve 801 under the seat 102a of the chair 102 also shields it from the lingering aerosol cloud thus reducing the exposure to aerosols. Under the dental chair mount with an on/off strong tape 806 or other releasable mount will provide stability to the whole setup. Also, using the on/off adhesive tape 806 causes no damage or possibility of voiding a warranty to a dental chair 102 and is reversable. Using this flexible sleeve 801 makes it always ready to put into use. A dentist can leave a flexible sleeve 801 mounted under each dental chair 102 and just move the connection to the vacuum collection canister 203 and or the tubing 201 to the dental funnel/filter. If using the HVE valve 701 in the cradle 706, then the dentist can just move the tubing needed 201,802 to set up a full line in any room. Alternately, each treatment room can have its own tubing 201,802 so that there are less parts to move thus saving time and expense. This configuration is important because not every treatment room has the same setup and may need a different length to accommodate the special needs of that room. Dentists often replace dental chairs with different life cycles of dental practice. This sectioning of the setup will allow a dentist to replace only the tubing 201,802 section with a longer or short part and still maintain the other parts that are functioning well. The present system allows for lightweight, less robust tubing to transport aerosols and thus requires less supporting elements to build the aerosol reduction system. The present system allows for use of all disposable tubing including, but not limited to paper or cardboard tubing and/or a polymeric material. The present system is not locked into more robust tubing required for transporting both liquid and air, since only air and aerosols are transporting by the tubing of the present development. The system can comprise both robust fixed tubing or all light weight disposable tubing or any combination thereof to best fit different dental units and age of dental units which allows for more of a custom fit to different situations in dental treatment rooms. Use of the dental assistant chair mounting of tubing as described herein provides for closer proximity to vacuum collection canister 203 and or HVE valve 701 in its cradle 706. The dental assistant chair 1000 has not heretofore been placed in any system of any self-supporting tubing or other tubing for a dental aerosol reduction system.

In the preceding specification, various embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

1. A self-supporting tube to support an associated medical or dental device, such as an extra-oral aerosol suction device.

2. The self-supporting tube as set forth in claim 1, configured as a high-volume evacuator tube for a dental chair system.

3. The self-supporting tube as set forth in claim 1, wherein the aerosol suction device includes a particle capturing media.

4. The self-supporting tube as set forth in claim 1, wherein the self-supporting tube includes a first end that connects physically and fluidically to an HVE port of a vacuum collection cannister and/or to an inlet of a high-volume evacuator valve, and wherein the self-supporting tube includes an opposite second end that connects physically and fluidically to the associated device such as the aerosol suction/filter device, and wherein the self-supporting tube includes at least one lumen that fluidically connects the first and second ends.

5. The self-supporting tube as set forth in claim 4, further comprising a manually operated inline valve.

6. The self-supporting tube as set forth in claim 4, further comprising an anti-rotational device that prevents rotation of the self-supporting tube relative to an associated cradle in which said high-volume evacuator valve is situated.

7. The self-supporting tube as set forth in claim 1, provided as part of a modular system comprising at least one flexible tube segment and said self-supporting tube connected together.

8. The self-supporting tube as set forth in claim 7, wherein at least one of the flexible tube segment and the self-supporting tube are connected to an associated support structure of a dental delivery system unit by a flexible sleeve through which said self-supporting tube passes.

9. The self-supporting tube as set forth in claim 8, wherein said support structure of said dental delivery system unit comprises at least one of: (i) a dental patient chair; (ii) a dental assistant's chair; (iii) a dentist chair; (iv) a lighting unit; (v) a cart or table; (vi) an imaging unit.

10. The self-supporting tube as set forth in claim 1, connected to an associated support structure of a dental system delivery unit by a flexible sleeve through which said self-supporting tube passes.

11. The self-supporting tube as set forth in claim 10, wherein said support structure comprises at least one of: (i) a dental patient chair; (ii) a dental assistant's chair; (iii) a dentist chair; (iv) a lighting unit; (v) a cart or table; (vi) an imaging unit.

12. The self-supporting tube as set forth in claim 1, wherein said tube is made entirely from a disposable material.

13. The self-supporting tube as set forth in claim 12, wherein said tube comprises: (i) a first end adapted to be connected to a suction source such as a vacuum collection cannister or a high-volume evacuator valve; and (ii) a second end that comprises the associated extra-oral aerosol suction device, wherein said extra-aerosol suction device is defined from paper or cardboard.

14. The self-supporting tube as set forth in claim 13, wherein said extra-aerosol suction device is constructed as a one-piece structure with said tube.

15. The self-supporting tube as set forth in claim 1, further comprising an adhesive or other fastening element on an exterior surface for adhering said tube to at least one of: (i) a dental patient chair; (ii) a dental assistant's chair; (iii) a dentist chair.

16. The self-supporting tube as set forth in claim 15, wherein said tube comprises a flat exterior surface on which said adhesive or other fastening element is located.

17. The self-supporting tube as set forth in claim 1, wherein said tube comprises a non-circular cross-section with at least one flat exterior surface on which an adhesive or other fastening element is located for selectively connecting the tube to at least one of: (i) a dental patient chair; (ii) a dental assistant's chair; (iii) a dentist chair.

18. The self-supporting tube as set forth in claim 1, further comprising a vacuum collection cannister and a U-shaped connector tube fitting comprising a first end connected to a port of said vacuum collection cannister and a second end connected to a first end of said self-supporting tube, wherein an opposite second end of said self-supporting tube includes an extra-oral aerosol suction device.

19. The self-supporting tube as set forth in claim 1, comprising a first end of the self-supporting tube connected to an inlet of a high-volume evacuator valve that is supported in a cradle and comprising an extra-oral aerosol suction device located at a second end of said self-supporting tube.

20. The self-supporting tube as set forth in claim 19, further comprising an anti-rotation device that is operably engaged between the self-supporting tube 201 and the cradle to prevent undesired rotation or movement of the self-supporting tube.

21. The self-supporting tube as set forth in claim 1, connected to a support arm that projects outwardly from a stopper that is non-rotatably seated in an HVE valve cradle.

22. The self-supporting tube as set forth in claim 1, provided as part of a modular system including a tube segment supported on a dental patient's chair or a dental assistant's chair.

23. The self-supporting tube as set forth in claim 22, wherein said tube segment includes first and second fittings on opposite first and second ends thereof.

24. The self-supporting tube as set forth in claim 23, further comprising first and second end caps that are releasably engaged with the first and second fittings of said tube segment.
